(19) United States
(12) Patent Application Publication
(10) Pub. No.: US 2016/0067340 A1
(43) Pub. Date: Mar. 10, 2016

(54) SEMI-SOLID CHEWABLE DOSAGE FORM FOR OVER-THE-COUNTER MEDICATIONS AND METHOD FOR PRODUCING SAME

(71) Applicant: Santa Cruz Pharmaceuticals, Inc., Santa Cruz, CA (US)

(72) Inventors: Michael T. WESTHUSING, Alamo, CA (US); Yong BAI, Santa Cruz, CA (US); Mario W. MEDRI, Miami Lakes, FL (US)

(21) Appl. No.: 14/626,897

(22) Filed: Feb. 19, 2015

Related U.S. Application Data
(60) Provisional application No. 62/046,712, filed on Sep. 5, 2014.

(51) Int. Cl. A61K 47/42; A61K 31/137; A61K 47/22; A61K 47/12; A61K 47/36; A61K 31/4402; A61K 47/26

(52) U.S. Cl. CPC A61K 47/42; A61K 31/4402; A61K 31/137; A61K 47/26; A61K 47/12; A61K 47/36; A61K 47/22

(57) ABSTRACT

The invention provides a semi-solid chewable dosage form that contains one or more active pharmaceutical ingredients that are generally available as over-the-counter medications including, for example, chlorpheniramine maleate, phenylephrine hydrochloride, guaifenesin, dextromethorphan hydrobromide, loratadine, or a combination thereof. The invention further provides a semi-solid chewable dosage form that contains chlorpheniramine maleate, phenylephrine hydrochloride or a combination thereof, a gelling agent, gelatin, sugar, a polyol, and a pH adjusting agent. The invention further provides a semi-solid chewable dosage form that contains the active pharmaceutical ingredient chlorpheniramine maleate, phenylephrine hydrochloride or a combination thereof, a gelling agent, gelatin, sugar, corn syrup, and a pH adjusting agent. The semi-solid chewable dosage form is useful for administration to individuals to treat symptoms from allergies, colds, congestion, and the like.

### CROSS-REFERENCE TO RELATED APPLICATIONS

This patent application claims the benefit of U.S. Provisional Application No. 62/045,712, filed Sep. 5, 2014, which is incorporated herein by reference in its entirety for all purposes.

### BACKGROUND OF THE INVENTION

Over-the-counter (OTC) medications are commonly used to treat various symptoms associated with allergies as well as colds. Chlorpheniramine maleate and phenylephrine hydrochloride are commonly used OTC drugs that have antihistamine and decongestant properties, respectively, that provide relief to individuals.

OTC medications are available in a variety of solid dosage forms that are taken orally including tablets, capsules, and soft-gels. The oral administration of solid dosage forms is difficult for some individuals who have difficulties swallowing any type of pills. This problem is magnified for solid dosage forms that need to be taken 2-4 times per day to provide the desired therapeutic effect. Solid dosage forms have an unpleasant after-taste. In addition, the need for a source of water or other liquid to assist with swallowing solid dosage forms can complicate administration.

As an alternative to solid dosage forms, OTC medications are also supplied as liquid suspensions or solutions to be taken orally. These liquid dosage forms are useful for administration to children. However, liquid dosages forms containing OTC medications often have a bitter taste from the active ingredients and other excipients present in the formulation. Additionally, the stability of such formulations over time can be a problem as active ingredients can degrade when either suspended or dissolved in a liquid medium.

The need remains for alternative dosage forms for OTC medications, in particular formulations containing chlorpheniramine maleate, phenylephrine hydrochloride, guaifenesen, dextromethorphan hydrobromide and loratadine, that have sufficient stability to be stored at room temperature for an extended duration and are further suitable for oral administration without an unpleasant taste or problem with swallowing.

### BRIEF SUMMARY OF THE INVENTION

An embodiment of the invention provides a semi-solid chewable dosage form that contains an active pharmaceutical ingredient, a gelling agent, gelatin, sugar, a polyol, and a pH adjusting agent. The active pharmaceutical ingredient may be chlorpheniramine maleate, phenylephrine hydrochloride, guaifenesin, dextromethorphan hydrobromide, loratadine, or a combination thereof.

In another embodiment, the invention provides a semi-solid chewable dosage form that contains an active pharmaceutical ingredient, a gelling agent, gelatin, sugar, corn syrup, and a pH adjusting agent. The active pharmaceutical ingredient may be chlorpheniramine maleate, phenylephrine hydrochloride, guaifenesin, dextromethorphan hydrobromide, loratadine, or a combination thereof.

A method of producing a semi-solid chewable dosage form is provided. The method comprises forming a primary blend comprising a gelling agent, sugar, a polyol and a pH adjusting agent, cooking the primary blend to obtain a residual moisture content to between 5% by weight to 25% by weight, combining the primary blend with a secondary blend containing an active pharmaceutical ingredient to yield a final blend, depositing the final blend into individual semi-solid chewable dosage forms. The active pharmaceutical ingredient may be chlorpheniramine maleate, phenylephrine hydrochloride, guaifenesin, dextromethorphan hydrobromide, loratadine, or a combination thereof.

The semi-solid chewable dosage form according to the invention is useful for administration to individuals, include both adults and children, to treat symptoms from allergies, colds and the like.

Other embodiments, characteristics, and advantages of the invention are apparent after reading the descriptions and examples that follow.

### DETAILED DESCRIPTION OF THE INVENTION

The semi-solid chewable dosage form of the invention (also referred to as the semi-solid dosage form) is intended to be chewed by a patient such that it is broken up into smaller parts within the oral cavity and then easily swallowed. The semi-solid dosage form has a sufficiently high viscosity that it is not pourable and further does not flow or conform to its container at room temperature. Typically, the semi-solid dosage form does not flow at low shear stress and generally exhibits plastic flow behavior. In general, the consistency of the semi-solid dosage form is the same as or similar to gelatin-based or pectin-based candy products such as, for example, gummy bears and pectin jellies.

The dosage form can have any size and shape such that it can be administered orally and chewed by a patient. The patient should be able to readily break apart the dosage form by chewing and further and swallow the dosage form without the need for an external source of liquid. Typically, the dosage form has a length of about 1 cm to about 5 cm, width of about 1 cm to about 5 cm and a height of about 1 cm to about 5 cm. Suitable shapes include, for example, ovals, spheres, cylinders, rectangular boxes and cubes. For administration to children (e.g., under the age of 13), the dosage form may be formed into figures including, for example, animals.

Generally, each individual dosage form has a total weight of at least 100 mg. Typically, each dosage form has a total weight of from about 1 g to about 20 g. Preferably, each dosage form has a total weight of from about 1 g to about 15 g. Preferably each dosage form has a total weight of from about 1 g to about 10 g, for example, about 1 g to about 1.5 g, about 1.5 g to about 2 g, about 2 g to about 2.5 g, about 2.5 g to about 3 g, about 3.5 g to about 4 g, about 4 g to about 4.5 g, about 4.5 g to about 5 g, about 5 g to about 5.5 g, about 5.5 g to about 6 g, about 6 g to about 6.5 g, about 6.5 g to about 7 g, about 7 g to about 7.5 g, about 7.5 g to about 8 g, about 8 g to about 8.5 g, about 8.5 g to about 9 g, about 9 g to about 9.5 g, and about 9.5 g to about 10 g. Most preferably, each dosage form has a total weight of about 5 g.

The semi-solid chewable dosage form of the invention includes one or more active pharmaceutical ingredients that are generally available as over-the-counter medications. Suitable active pharmaceutical ingredients include, for example, chlorpheniramine maleate, phenylephrine hydrochloride, guaifenesin, dextromethorphan hydrobromide, loratadine, or a combination thereof. In one embodiment, the dosage form contains a combination of chlorpheniramine maleate and phenylephrine hydrochloride. In another embodiment, the dosage form contains a combination of dextromethorphan hydrobromide and phenylephrine hydrochloride.

Chlorpheniramine maleate is a pharmaceutically acceptable salt of chlorpheniramine. Chlorpheniramine has the following chemical structure:

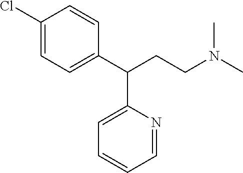 [SMILES: CN(C)CCC(c1ccc(Cl)cc1)c1ccccn1]

In some embodiments, the amount of chlorpheniramine maleate present in each dosage form is from about 0.1 mg to about 30 mg. Preferably, the amount of chlorpheniramine maleate present in each dosage form is from about 1 mg to about 10 mg. More preferably, the amount of chlorpheniramine maleate present in each dosage form is about 1 mg to about 5 mg. Most preferably, the amount of chlorpheniramine present is about 2 mg or about 4 mg in each dosage form that has a total weight of about 5 g.

Alternatively, chlorpheniramine maleate may be present in the dosage form in amount from about 0.01% by weight to about 1.0% by weight, and preferably about 0.02% to about 0.2% by weight. For an adult dose, chlorpheniramine maleate is preferably present in an amount from about 0.06% by weight to about 0.1% by weight. For a pediatric dose (e.g., children under 13), chlorpheniramine maleate is preferably present in an amount from about 0.03% by weight to about 0.05% by weight.

Phenylephrine hydrochloride is a pharmaceutically acceptable salt of phenylephrine. Phenylephrine has the following chemical structure:

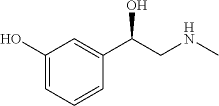

In some embodiments, the amount of phenylephrine hydrochloride present in each dosage form is from about 0.1 mg to about 20 mg. Preferably, the amount of phenylephrine hydrochloride present is from about 2 mg to about 15 mg. More preferably, the amount of phenylephrine hydrochloride present is from about 3 mg to about 12 mg. Most preferably, the amount of phenylephrine hydrochloride present is about 5 mg or about 10 mg in each dosage form that has a total weight of about 5 g.

Alternatively, phenylephrine hydrochloride may be present in the dosage form in amount from about 0.01% by weight to about 1% by weight, and preferably 0.01% to about 0.5% by weight. For an adult dose, phenylephrine hydrochloride is preferably present in an amount from about 0.15% by weight to about 0.25% by weight. For a pediatric dose (e.g., children under 13), phenylephrine hydrochloride is preferably present in an amount from about 0.05% by weight to about 0.15% by weight.

Guaifenesin has the following chemical structure:

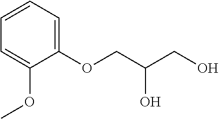

In some embodiments, the amount of guaifenesin present in each dosage form is from about 10 mg to about 1,500 mg. Preferably, the amount of guaifenesin present in each dosage form is from about 200 mg to about 1,200 mg. More preferably, the amount of guaifenesin present in each dosage form is about 100 mg to about 400 mg. Most preferably, the amount of guaifenesin present is about 100 mg, 200 mg or about 400 mg in each dosage form that has a total weight of about 5 g.

Alternatively, guaifenesin may be present in the dosage form in amount from about 0.1% by weight to about 20% by weight, and preferably about 0.5% to about 10% by weight. For an adult dose, guaifenesin is preferably present in an amount from about 0.5% by weight to about 5% by weight. For a pediatric dose (e.g., children under 13), guaifenesin is preferably present in an amount from about 0.1% by weight to about 4% by weight.

Dextromethorphan hydrobromide is a pharmaceutically acceptable salt of dextromethorphan. Dextromethorphan has the following chemical structure:

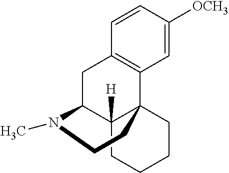

In some embodiments, the amount of dextromethorphan hydrobromide present in each dosage form is from about 1 mg to about 100 mg. Preferably, the amount of guaifenesin present in each dosage form is from about 5 mg to about 60 mg. More preferably, the amount of guaifenesin present in each dosage form is about 10 mg to about 30 mg. Most preferably, the amount of guaifenesin present is about 10 mg or about 20 mg in each dosage form that has a total weight of about 5 g.

Alternatively, dextromethorphan hydrobromide may be present in the dosage form in amount from about 0.01% by weight to about 2% by weight, and preferably about 0.1% to about 1% by weight. For an adult dose, guaifenesin is preferably present in an amount from about 0.1% by weight to about 1% by weight. For a pediatric dose (e.g., children under 13), guaifenesin is preferably present in an amount from about 0.1% by weight to about 0.8% by weight.

Loratadine has the following chemical structure:

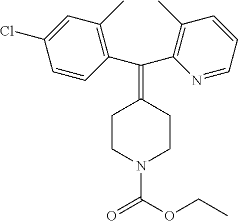 [SMILES: CCOC(=O)N1CCC(=C(c2ccc(Cl)cc2C)c2ncccc2C)CC1]

In some embodiments, the amount of loratadine present in each dosage form is from 1 mg to about 100 mg. Preferably, the amount of loratadine present in each dosage form is from about 5 mg to about 50 mg. More preferably, the amount of loratadine present in each dosage form is from about 10 mg to about 30 mg. Most preferably, the amount of loratadine present is about 10 mg in each dosage form that has a total weight of about 5 g.

Alternatively, loratadine may be present in the dosage form in amount from about 0.01% by weight to about 2% by weight, and preferably about 0.1% to about 1% by weight. For an adult dose, loratadine is preferably present in an amount from about 0.1% by weight to about 1% by weight. For a pediatric dose (e.g., children under 13), loratadine is preferably present in an amount from about 0.1% by weight to about 0.5% by weight.

In embodiments in which chlorpheniramine maleate and phenylephrine hydrochloride are both present in the dosage form, preferably chlorpheniramine maleate is present in an amount of about 2 mg and phenylephrine hydrochloride is present in an amount of about 5 mg. Alternatively, chlorpheniramine maleate is present in an amount of about 4 mg and phenylephrine hydrochloride is present in an amount of about 10 mg. Typically, a pediatric dose contains about 2 mg chlorpheniramine maleate and 5 mg phenylephrine hydrochloride and an adult dose contains about 4 mg chlorpheniramine maleate and 10 mg phenylephrine hydrochloride.

In embodiments in which dextromethorphan hydrobromide and phenylephrine hydrochloride are both present in the dosage form, preferably dextromethorphan hydrobromide is present in an amount of about 10 mg and phenylephrine hydrochloride is present in an amount of about 5 mg. Alternatively, dextromethorphan hydrobromide is present in an amount of about 20 mg and phenylephrine hydrochloride is present in an amount of about 10 mg.

Other active pharmaceutical ingredients suitable for use as an OTC medication in the semi-solid dosage form for the invention include, by way of example, antihistamines, antitussives, decongestants, expectorants, analgesics, anti-inflammatories, and/or anti-GERD medications. In particular, the active pharmaceutical ingredient may be pseudoephedrine, diphenhydramine, codeine, desloratadine, fexofenadine, ranitidine, cimetidine, famotidine, omeprazole, esomeprazole, lansoprazole and pharmaceutically acceptable salts thereof. Combinations of two or more active pharmaceutical ingredients may be used in the semi-solid dosage form of the invention.

The amount of active pharmaceutical ingredient present for use as an OTC medication in the semi-solid dosage form will vary for each different active. Typically, the semi-solid dosage form contains about 0.1 mg to 1 g of the active pharmaceutical ingredient. Alternatively, the semi-solid dosage form contains one or more active pharmaceutical ingredients in an amount from about 0.01% by weight to about 10% by weight.

The semi-solid dosage form of the invention may be administered once per day or multiple times per day to provide relief for various symptoms affecting an individual. For example, chlorpheniramine maleate may be administered to treat symptoms of allergic rhinitis or sinusitis. Phenylephrine hydrochloride may be administered to treat symptoms of nasal congestion. Typical dosing of chlorpheniramine maleate for adults is 4 mg every 4-6 hours and for children (i.e., 6-11 years old) is 2 mg every 4-6 hours. Typical dosing of phenylephrine hydrochloride for adults is 10 mg every 4-6 hours and for children (i.e., 6-11 years old) is 5 mg every 4-6 hours.

Guaifenesin may be administered to treat symptoms of congestion in the chest and throat. Typical dosing of guaifenesin for adults is 200 mg to 400 mg every 4-6 hours and for children (i.e., 6-11 years old) is 100 mg to 200 mg every 4-6 hours.

Dextromethorphan hydrobromide may be administered to treat symptoms of a cough. Typical dosing of dextromethorphan hydrobromide for adults is 10 mg to 30 mg every 4-8 hours and for children (i.e., 6-11 years old) is 5 mg to 10 mg every 4 hours.

Loratadine may be administered to treat symptoms of allergic rhinitis and urticaria. Typical dosing of loratadine for adults and children (i.e., 6-11 years old) is 10 mg per day.

The semi-solid dosage form of the invention includes a gelling agent. Any suitable gelling agent may be used to provide the dosage form with the desired characteristics including, for example, semi-solid structure, shape and texture. The gelling agent is typically a USP (U.S. Pharmacopeia) grade gelling agent. Preferably, the gelling agent is pectin.

Pectin is a purified carbohydrate obtained by aqueous extraction from citrus peel or apple pomace. Any suitable type of pectin may be use in the dosage form including, for example, high-methoxy pectin and low-methoxy pectin and combinations thereof. Low-methoxy pectin may be amidated which is often referred to as LMA pectin. Examples of suitable pectins are Genu® citrus pectin USP/100 and Genu® citrus pectin USP/200 from CP Kelco.

Pectin may be generally present in the semi-solid dosage form in an amount of from about 0.01% by weight to about 10% by weight. Preferably, pectin is present in an amount of from about 0.5% by weight to about 7% by weight, for example from about 0.5% to about 1%, from about 1% to about 1.5%, from about 1.5% to about 2%, from about 2% to about 2.5%, from about 2.5% to about 3%, from about 3% to about 3.5%, from about 3.5% to about 4%, from about 4% to about 4.5%, from about 4.5% to about 5%, from about 5% to about 5.5%, from about 5.5% to about 6%, from about 6% to about 6.5%, and from about 6.5% to about 7%. More preferably, pectin is present in an amount from about 1% by weight to about 5% by weight.

The semi-solid dosage form of the invention includes gelatin. Without being bound by any theory, it is believed that the presence of gelatin assists with gelling of the semi-solid dosage form and further serves to mask the taste of the active ingredients.

Any suitable type of gelatin may be present in the dosage form. For example, the gelatin may be animal-derived gelatin, chemically-modified gelatin, physically-modified gelatin, and combinations thereof. Animal-derived gelatin may be derived from any suitable source such as, for example, pigskin or bovine bone.

Alternatively, the gelatin may be hydrolyzed gelatin. Hydrolyzed gelatin is also commonly known as hydrolyzed collagen, collagen hydrolysate, and collagen peptide. Hydrolyzed gelatin having a molecular weight ranging from about 2,500 to about 5,000 may be used. An example of a suitable hydrolyzed gelatin is Peptiplus® powder from Gelita.

Gelatin may be generally present in the semi-solid dosage form in an amount from about 0.01% by weight to about 15% by weight. Preferably, gelatin is present in an amount of from about 0.5% by weight to about 8% by weight, for example from about 0.5% to about 1%, from about 1% to about 1.5%, from about 1.5% to about 2%, from about 2% to about 2.5%, from about 2.5% to about 3%, from about 3% to about 3.5%, from about 3.5% to about 4%, from about 4% to about 4.5%, from about 4.5% to about 5%, from about 5% to about 5.5%, from about 5.5% to about 6%, from about 6% to about 6.5%, from about 6.5% to about 7%, from about 7% to about 7.5%, and from about 7.5% to about 8%. More preferably, gelatin is present in an amount from about 1% by weight to about 5% by weight.

The semi-solid dosage form of the invention includes sugar. Generally, sugar is present in an amount from about 30% by weight to about 99% by weight of the dosage form. Preferably, sugar is present in an amount from about 40% by weight to about 95% by weight, for example, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, and from about 90% to about 85%.

In some embodiments of the invention, the semi-solid dosage form includes a polyol. Polyols are also referred to as sugar alcohols. Without being bound by any theory, the presence of a polyol is believed to promote the stability of the semi-solid dosage form of the invention.

Suitable polyols include, for example, hydrogenated starch hydrolysates, isomalt, lactitol, maltitol, mannitol, sorbitol, erythritol, and xylitol. Combinations of polyols may be used. Preferably, the polyol is hydrolyzed starch hydrolysates (HSH). HSH typically contains substantial quantities of hydrogenated oligo- and poly-saccharides in addition to monomeric and dimeric polyols. HSH is commonly known to include polyglycitol. An example of a commercially available HSH is Hystar® 3375 syrup (75% solids), Hystar® 4075 and Hystar® 6075 supplied by SPI Polyols. Other commercially available HSH include 75/400 from Roquette and Stabilite® liquid HSH and Stabilite® powdered HSH supplied by Corn Products Specialty Ingredients.

One or more polyols may be present in the semi-solid dosage form in an amount from about 30% by weight to about 99% by weight. Preferably, one or more polyols may be present in an amount from about 40% by weight to about 90% by weight, for example, about 40% to about 50%, about 50% to about 60%, about 60% to about 70%, about 70% to about 80%, and about 80% to about 90%.

In embodiments in which one or more polyols are present, the ratio of polyol to sugar is typically from about 1:10 to about 10:1 by dry weight. Preferably the ratio of polyol to sugar is from about 1:2 to about 2:1 by dry weight, for example, from about 1:1.5 to about 1:5.1.

In some embodiments, the semi-solid dose form includes corn syrup. Corn syrup may be present without a polyol. Alternatively, corn syrup may be present in addition to a polyol. Any suitable corn syrup may be used, for example, corn syrup having 36-65 DE (dextrose equivalents), preferably corn syrup 42-43 DE. Corn syrup may contain about 50% by weight to about 90% by weight solids, preferably about 80% solids.

Corn syrup may be present in the semi-solid dosage form in an amount from about 30% by weight to about 99% by weight. Preferably, corn syrup may be present in an amount from about 40% by weight to about 90% by weight, for example, about 40% to about 50%, about 50% to about 60%, about 60% to about 70%, about 70% to about 80%, and about 80% to about 90%.

In embodiments in which corn syrup is present, the ratio of corn syrup to sugar is typically from about 1:10 to about 10:1 by dry weight. Preferably the ratio of corn syrup to sugar is from about 1:2 to about 2:1 by dry weight, for example, from about 1:1.5 to about 1:5.1.

The semi-solid dosage form includes a pH adjusting agent. Any suitable pH adjusting agent may be used that is sufficient to adjust the pH during the manufacture of the dosage form. By way of example, the pH adjusting agent may be sodium citrate, citric acid, sodium ascorbate and ascorbic acid. Two or more pH adjusting agents may be used. The pH adjusting agent may be supplied in solid form (e.g., as a powder) or in aqueous solution. For example, citric acid may be supplied in a 50% solution. Preferably, the pH adjusting agent is sodium citrate or citric acid. More preferably, both sodium citrate and citric acid are included in the semi-solid dosage form as pH adjusting agents.

The pH adjusting agent may be present in the semi-solid dosage form in an amount from about 0.1% by weight to about 5% by weight. Preferably, the pH adjusting agent may be present in an amount from about 1% to about 5% by weight, for example, from about 1% to about 1.5%, from about 1.5% to about 2%, from about 2% to about 2.5%, from about 2.5% to about 3%, from about 3% to about 3.5%, from about 3.5% to about 4.0%, from about 4% to about 4.5%, and from about 4.5% to about 5%.

In some embodiments, sodium citrate is present in an amount from about 0.1% by weight to about 1% by weight. Preferably, sodium citrate is present in an amount from about 0.1% by weight to about 0.5% by weight, for example, from about 0.1% to about 0.2%, from about 0.2% to about 0.3%, from about 0.3% to about 0.4%, and from about 0.4% to about 0.5%.

In other embodiments, citric acid is present (as 50% aqueous solution) in an amount from about 0.5% by weight to about 3% by weight, for example from about 0.5% to about 1%, from about 1% to about 1.5%, from about 1.5% to about 2%, from about 2% to about 2.5%, and from about 2.5% to about 3%.

In certain embodiments, the semi-solid dosage form contains glycerin, also commonly known as glycerol. Without being bound by any theory, glycerin is believed to function as an emollient to stability the dosage form during its preparation. Preferably, glycerin USP is used. Glycerin may be present in the semi-solid dosage form in an amount from about 0.1% by weight to about 10% by weight. Preferably, glycerin is present in an amount from about 0.5% by weight to about 5% by weight, for example from about 0.5% to about 1%, from about 1% to about 1.5%, from about 1.5% to about 2.0%, from about 2.0% to about 2.5%, from about 2.5% to about 3.0%, from about 3.0% to about 3.5%, from about 3.5% to about 4.0%, from about 4.0% to about 4.5%, and from about 4.5% to about 5.0%.

In some embodiments, the semi-solid dosage form contains a flavorant. Any suitable food-grade flavorant may be used to suppress the bitterness of the active ingredients to provide a pleasant taste to the dosage form upon chewing and swallowing. A mixture of two or more flavorants may be used to yield the desired taste characteristic.

Suitable flavorants include artificial sweeteners such as, for example, sucralose, acesulfame potassium, stevia, sodium saccharine, erythritol, and aspartame. Another suitable flavorant may be a fraction of the lactone group such as, for example, decalactone and dodecalactone (e.g., gamma dodecalactone). Lactone fractions are typically supplied in a propylene glycol solution, in particular from 0.5% to 1% in propylene glycol solution. The flavorant may be orange or cherry flavors. Alternatively, the flavorant may be menthol.

Preferably, the flavorant is an artificial sweetener. More preferably, the artificial sweetener is sucralose.

The flavorant may be present in an amount up to about 1% by weight, preferably up to about 0.5% by weight, for example, up to about 0.01%, up to about 0.05%, up to about 0.1%, up to about 0.2%, up to about 0.3%, up to about 0.4%, and up to about 0.5%. In certain embodiments, the amount of flavorant present is in a range bounded by any of the foregoing values. Fractions of the lactone group may be present in an amount of from about 1 ppm to 50 ppm, preferably from about 2 ppm to about 10 ppm, and more preferably from about 3 ppm to about 9 ppm.

A colorant may optionally be added to provide a suitable appearance for the semi-solid dosage form. Examples of suitable colorants include red or yellow dyes such as FD&C Red #40 and FD&C Yellow #6. Two or more colorants may be combined.

The semi-solid chewable dosage form of the invention generally has a water content, also referred to as a residual moisture content, of less than about 15% by weight, e.g., about 14% or less, about 13% or less, about 12% or less, about 11% or less, about 10% or less, about 9% or less, about 8% or less, about 7% or less, about 6% or less, or about 5% or less. In other embodiments, the water content of the semi-solid dosage form is in a range bounded by any of the foregoing values. Preferably, the water content of the semi-solid dosage form is from about 8% by weight to about 15% by weight.

In some embodiments, the semi-solid chewable dosage form comprises:

one or more active pharmaceutical ingredients in an amount from about 0.01% by weight to about 10% by weight;

pectin in an amount from about 0.5% by weight to about 7% by weight;

sugar in an amount from about 40% by weight to about 95% by weight;

hydrolyzed starch hydrolysate in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight, wherein the water content of the semi-solid dosage form is from about 8% by weight to about 15% by weight.

In some embodiments, the semi-solid chewable dosage form comprises:

one or more active pharmaceutical ingredients in an amount from about 0.01% by weight to about 10% by weight;

pectin in an amount from about 0.5% by weight to about 7% by weight;

sugar in an amount from about 40% by weight to about 95% by weight;

corn syrup in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight, wherein the water content of the semi-solid dosage form is from about 8% by weight to about 15% by weight.

In some embodiments, the semi-solid chewable dosage form comprises:

chlorpheniramine maleate;

phenylephrine hydrochloride;

pectin in an amount from about 0.5% by weight to about 7% by weight;

sugar in an amount from about 40% by weight to about 95% by weight;

hydrolyzed starch hydrolysate in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight, wherein the water content of the semi-solid dosage form is from about 8% by weight to about 15% by weight.

In some embodiments, the semi-solid chewable dosage form comprises:

chlorpheniramine maleate;

phenylephrine hydrochloride;

pectin in an amount from about 0.5% by weight to about 7% by weight;

sugar in an amount from about 40% by weight to about 95% by weight;

corn syrup in an amount from about 40% by weight to about 90% by weight;

hydrolyzed gelatin in an amount from about 0.5% by weight to about 8% by weight;

sodium citrate in an amount from about 0.1% by weight to about 1% by weight; and

citric acid in an amount from about 0.5% by weight to about 3% by weight, wherein the water content of the semi-solid dosage form is from about 8% by weight to about 15% by weight.

The semi-solid chewable dosage form of the invention can be prepared by any suitable method including, for example, a batch process or a continuous process. In some embodiments, the components of the dosage form are first combined together in a suitable vessel. The components can be combined in any suitable order.

During manufacturing, water is typically added to the combination of some or all of the components to form a mixture that is the base for the semi-solid dosage form. In some embodiments, pectin, sugar, a polyol, and a pH adjusting agent are combined with water to form the base. Alternatively, pectin, sugar, corn syrup, and a pH adjusting agent are combined with water to form the base. Any amount of water may be added to prepare a suitable mixture. In some embodiments, a sufficient amount of water is added to dissolve water-soluble components, for example, sugar, and uniformly disperse non-water-soluble components to form a mixture.

Following the preparation of the base containing the components of the semi-solid dosage form along with water, the base typically has a water content of from about 10% by weight to about 90% by weight. Preferably, the base has a water content of from about 20% by weight to about 50% by weight, for example, about 20% to about 25%, about 25% to about 30%, about 30% to about 35%, about 35% to about 40%, about 40% to about 45%, and about 45% to about 50%.

In some embodiments, the base is cooked at a suitable temperature to remove a portion of the water present. By reducing the water content through cooking, the base may be converted into a semi-solid chewable dosage form having the desired physical characteristics, in particular consistency and texture. The base may be cooked by any suitable means including, for example, with a steam-jacketed vessel or a conventional heat exchanger. Cooking may optionally be carried out with the aid of a vacuum.

The base may be cooked at any suitable temperature and for a sufficient length of time to yield a molten mass having the desired water content. Generally, following cooking, the base has a residual moisture content from about 5% by weight to about 25% by weight. Preferably, the base has a residual moisture content after cooking from about 9% by weight to about 20% by weight, for example, about 9% to about 10%, about 10% to about 11%, about 11% to about 12%, about 12% to about 13%, about 13% to about 14%, and about 14% to about 15%, about 15% to about 16%, about 16% to about 17%, about 17% to about 18%, about 18% to about 19%, and about 19% to about 20%. In certain aspects, the residual moisture content of the base after cooking is an amount to provide a semi-solid dosage form containing about 0.01% by weight to about 2% by weight of the active ingredients.

Generally, the base is cooked at a temperature of from about 220° F. to about 265° F. Preferably, the base may be cooked at a temperature of about 230° F. to about 250° F., for example, about 230° F. to about 235° F., about 235° F. to about 240° F., about 240° F. to about 245° F., and about 245° F. to about 250° F.

After the base is cooked for a sufficient time to yield a molten mass, any remaining components of the semi-solid dosage form may be added such as, for example, the active pharmaceutical ingredients chlorpheniramine maleate and phenylephrine hydrochloride, hydrolyzed gelatin, glycerin, a flavorant, and a colorant to form the final blend. These additional components may be added to the base by any suitable means using, for example, mass flow meters and static mixers.

A pH adjusting agent, such as citric acid, may be added to the base to provide a suitable pH for the final blend that contains all of the components of the semi-solid dosage form. The pH of the final blend is generally from about 4 to about 6, preferably from about 4.5 to about 5.5.

In some embodiments, different blends of components are prepared separately and then combined together to form a final blend from which the semi-solid dosage form is obtained. For example, a primary blend may be combined with a secondary blend to form the final blend. A separate blend containing flavorants and/or colorants and an acid solution may optionally be added in the preparation of the final blend.

In one embodiment, a primary blend is prepared by combining pectin, sugar, a polyol, and a pH adjusting agent with water. Alternatively, the primary blend may be prepared by combining pectin, sugar, corn syrup, and a pH adjusting agent with water. The amount of water and corn syrup. A pH adjusting agent such as, for example, sodium citrate may optionally be added to the primary blend. In some embodiments, the primary blend has a pH from about 2 to about 6, preferably from about 2.5 to about 4, and more preferably from about 2.8 to about 3.8.

In certain aspects, the primary blend is cooked at an appropriate temperature and for an appropriate length of time to provide the primary blend with any suitable moisture content for further processing. Preferably, the primary blend has a moisture content after cooking from about 5% by weight to about 25% by weight. Preferably, the primary blend has a residual moisture content after cooking from about 9% by weight to about 20% by weight, for example, about 9% to about 10%, about 10% to about 11%, about 11% to about 12%, about 12% to about 13%, about 13% to about 14%, and about 14% to about 15%, about 15% to about 16%, about 16% to about 17%, about 17% to about 18%, about 18% to about 19%, and about 19% to about 20%. Generally, the primary blend may be cooked at a temperature of about 230° F. to about 250° F., for example, about 230° F. to about 235° F., about 235° F. to about 240° F., about 240° F. to about 245° F., and about 245° F. to about 250° F.

A secondary blend may be added to the primary blend after cooking is completed. The secondary blend may contain one or more components of the semi-solid dosage form. In some embodiments, the secondary blend includes chlorpheniramine maleate, phenylephrine hydrochloride, and hydrolyzed gelatin. Water may be added to the secondary blend to dissolve water-soluble components and/or form a homogenous mixture. Other components may be added to the secondary blend including, for example, glycerin, flavorants and colorants. Alternatively, an additional blend may be prepared containing glycerin, flavorants and colorants. An acid solution may further be prepared containing citric acid to obtain the desired pH of the final blend. The final blend may be obtained by combining the primary blend, secondary blend, additional blend and citric acid in any order.

The final blend may be further processed as needed prior to preparation of the semi-solid dosage form. For example, the final blend may be transferred to a depositor hopper having a jacket to maintain a temperature of from about 180° F. to about 210° F., preferably about 190° to about 200° F. After a suitable amount of time, the final blend may be dispensed from the depositor hopper to product the semi-solid chewable dosage form of the invention.

The semi-solid chewable dosage form may be obtained by depositing the final blend into pre-formed plastic molds using conventional techniques. Preferably, the plastic molds are blister packs having multiple cavities that provide for unit dose packaging of the semi-solid dosage form without having to transfer the dosage form from a mold to a separate container. The dosage form solidifies in the plastic molds which serve as the final packaging. As the temperature of the dosage form cools, the dosage form takes its final shape in the cavities of the blister pack. The blister pack is preferably sealed, for example, using foil. One or more blister packs may be packaged in containers. Alternatively, the dosage forms may be prepared in molds and transferred to other suitable containers.

Advantageously, a pre-determined amount of the final blend, for example based on weight, is dispensed into each cavity to form individual pieces. The individual pieces contain the desired amount of the active ingredients, for example 4 mg chlorpheniramine maleate and 10 mg phenylephrine hydrochloride for a adult dose and 2 mg chlorpheniramine maleate and 5 mg phenylephrine hydrochloride for a pediatric dose.

The following examples further illustrate the invention but, of course, should not be construed as in any way limiting its scope.

### Example 1

This example demonstrates a semi-solid chewable dosage form and its method of preparation in accordance with an embodiment of the invention. A 200 g batch is produced in this example. Each individual piece weighs 5 grams and contains 2 mg of chlorpheniramine maleate and 5 mg of phenylephrine hydrochloride.

| | Formula | Batch |
|---|---|---|
| Ingredient | % by weight | 200 g |
| Sugar (powder) | 40.17 | 80.34 |
| Corn Syrup (dry) | 41.43 | 82.86 |
| Sodium Citrate (powder) | 0.39 | 0.78 |
| USP Citrus Pectin 200 (high methoxy) | 1.97 | 3.94 |
| Residual Water | 11.13 | 22.26 |
| Chlorpheniramine Maleate | 0.04 | 0.08 |
| Phenylephrine Hydrochloride | 0.10 | 0.20 |
| Hydrolyzed Gelatin | 1.48 | 2.96 |
| Artificial sweetener (Sucralose) | 0.30 | 0.6 |
| Dodecalactone (1% in PG sol) | 0.01 | 0.02 |
| FD&C Yellow #6 | 0.01 | 0.02 |
| Glycerin USP | 1.97 | 3.94 |
| Orange Flavor FFS (211P52) | 0.20 | 0.40 |
| Menthol | 0.05 | 0.1 |
| Citric Acid (powder) | 0.75 | 1.5 |

A primary blend is prepared that contains sugar, corn syrup, sodium citrate, pectin and water. The primary blend is cooked to produce a residual moisture content of about 11% by weight. A secondary blend is prepared that contains chlorpheniramine maleate, phenylephrine hydrochloride, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 2

This example demonstrates a semi-solid chewable dosage form and its method of preparation in accordance with an embodiment of the invention. A 200 g batch is produced in this example. Each individual piece weighs 5 grams and contains 2 mg of chlorpheniramine maleate and 5 mg of phenylephrine hydrochloride.

| | Formula | Batch |
|---|---|---|
| Ingredient | % by weight | 200 g |
| Sugar (powder) | 42.75 | 85.50 |
| Hydrogenated Starch Hydrolysate (HSH) (dry) | 38.77 | 77.54 |
| Sodium Citrate (powder) | 0.35 | 0.70 |
| USP Citrus Pectin 200 (high methoxy) | 1.99 | 3.98 |
| Residual Water | 11.20 | 22.40 |
| Chlorpheniramine Maleate | 0.04 | 0.08 |
| Phenylephrine Hydrochloride | 0.10 | 0.20 |
| Hydrolyzed Gelatin | 1.49 | 2.98 |
| Artificial sweetener (Sucralose) | 0.30 | 0.6 |
| Dodecalactone (1% in PG sol) | 0.01 | 0.02 |
| FD&C Yellow #6 | 0.01 | 0.02 |
| Glycerin USP | 1.99 | 3.98 |
| Orange Flavor FFS (211P52) | 0.20 | 0.40 |
| Menthol | 0.05 | 0.10 |
| Citric Acid (powder) | 0.75 | 1.50 |

A primary blend is prepared that contains sugar, hydrogenated starch hydrolysate, sodium citrate, pectin and water. The primary blend is cooked to produce a residual moisture content of about 11% by weight. A secondary blend is prepared that contains chlorpheniramine maleate, phenylephrine hydrochloride, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 3

This example demonstrates a semi-solid chewable dosage form and its method of preparation in accordance with an embodiment of the invention. A 200 g batch is produced in this example. Each individual piece weighs 5 grams and contains 4 mg of chlorpheniramine maleate and 10 mg of phenylephrine hydrochloride.

| | Formula | Batch |
|---|---|---|
| Ingredient | % by weight | 200 g |
| Sugar (powder) | 40.12 | 80.24 |
| Corn Syrup (dry) | 41.37 | 82.74 |
| Sodium Citrate (powder) | 0.39 | 0.78 |
| USP Citrus Pectin 200 (high methoxy) | 1.97 | 3.94 |
| Residual Water | 11.10 | 22.20 |
| Chlorpheniramine Maleate | 0.08 | 0.16 |
| Phenylephrine Hydrochloride | 0.20 | 0.4 |
| Hydrolyzed Gelatin | 1.48 | 2.96 |
| Artificial sweetener (Sucralose) | 0.30 | 0.6 |
| Dodecalactone (1% in PG sol) | 0.01 | 0.02 |
| FD&C Red #40 | 0.01 | 0.02 |
| Glycerin USP | 1.97 | 3.94 |
| Cherry Flavor FFS (223G12) | 0.20 | 0.40 |
| Menthol | 0.05 | 0.1 |
| Citric Acid (powder) | 0.75 | 1.5 |

A primary blend is prepared that contains sugar, corn syrup, sodium citrate, pectin and water. The primary blend is cooked to produce a residual moisture content of about 11% by weight. A secondary blend is prepared that contains chlorpheniramine maleate, phenylephrine hydrochloride, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 4

This example demonstrates a semi-solid chewable dosage form and its method of preparation in accordance with an embodiment of the invention. A 200 g batch is produced in this example. Each individual piece weighs 5 grams and contains 4 mg of chlorpheniramine maleate and 10 mg of phenylephrine hydrochloride

| | Formula | Batch |
|---|---|---|
| Ingredient | % by weight | 200 g |
| Sugar (powder) | 42.69 | 85.38 |
| Hydrogenated Starch Hydrolysate (HSH) (dry) | 38.72 | 77.44 |
| Sodium Citrate (powder) | 0.35 | 0.70 |
| USP Citrus Pectin 200 (high methoxy) | 1.99 | 3.98 |
| Residual Water | 11.17 | 22.34 |
| Chlorpheniramine Maleate | 0.08 | 0.16 |
| Phenylephrine Hydrochloride | 0.20 | 0.40 |
| Hydrolyzed Gelatin | 1.49 | 2.98 |
| Artificial sweetener (Sucralose) | 0.30 | 0.6 |
| Dodecalactone (1% in PG sol) | 0.01 | 0.02 |
| FD&C Red #40 | 0.01 | 0.02 |
| Glycerin USP | 1.99 | 3.98 |
| Cherry Flavor FFS (223G12) | 0.20 | 0.40 |
| Menthol | 0.05 | 0.10 |
| Citric Acid (powder) | 0.75 | 1.50 |

A primary blend is prepared that contains sugar, hydrogenated starch hydrolysate, sodium citrate, pectin and water. The primary blend is cooked to produce a residual moisture content of about 11% by weight. A secondary blend is prepared that contains chlorpheniramine maleate, phenylephrine hydrochloride, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 5

This example demonstrates a semi-solid chewable dosage form and its method of preparation in accordance with an embodiment of the invention. A 200 g batch is produced in this example. Each individual piece weighs 5 grams and contains 200 mg of guaifenesin.

| | Formula | Batch |
|---|---|---|
| Ingredient | % by weight | 200 gr |
| Sugar (powder) | 34.00 | 68.00 |
| Hydrogenated Starch Hydrolysate (HSH) (dry) | 38.00 | 76.00 |
| Sodium Citrate (powder) | 0.40 | 0.80 |
| USP Citrus Pectin 200 (high methoxy) | 2.99 | 5.98 |
| Residual Water | 13.79 | 27.58 |
| Guaifenesin (USP) powder | 4.00 | 8.00 |
| Hydrolyzed Gelatin | 3.00 | 6.00 |
| Artificial sweetener (Sucralose) | 0.50 | 1.00 |
| Dodecalactone (1% in PG sol) | 0.02 | 0.04 |
| FD&C Red #40 | 0.01 | 0.02 |
| Glycerin USP | 1.99 | 3.98 |
| Cherry Flavor FFS (223G12) | 0.20 | 0.40 |
| Menthol | 0.10 | 0.20 |
| Citric Acid (powder) | 1.00 | 2.00 |

A primary blend is prepared that contains sugar, hydrogenated starch hydrolysate, sodium citrate, pectin and water. The primary blend is cooked to produce a residual moisture content of about 14% by weight. A secondary blend is prepared that contains guaifenesin, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 6

This example demonstrates a semi-solid chewable dosage form and its method of preparation in accordance with an embodiment of the invention. A 200 g batch is produced in this example. Each individual piece weighs 5 grams and contains 10 mg of dextromethorphan HBr and 5 mg of phenylephrine hydrochloride.

| | Formula | Batch |
|---|---|---|
| Ingredient | % by weight | 200 g |
| Sugar (powder) | 40.10 | 80.20 |
| Corn Syrup (dry) | 41.37 | 82.74 |
| Sodium Citrate (powder) | 0.39 | 0.78 |
| USP Citrus Pectin 200 (high methoxy) | 1.97 | 3.94 |
| Residual Water | 11.10 | 22.20 |
| Dextromethorphan Hydrobromide | 0.20 | 0.40 |
| Phenylephrine Hydrochloride | 0.10 | 0.20 |
| Hydrolyzed Gelatin | 1.48 | 2.96 |
| Artificial sweetener (Sucralose) | 0.30 | 0.6 |
| Dodecalactone (1% in PG sol) | 0.01 | 0.02 |
| FD&C Red #40 | 0.01 | 0.02 |
| Glycerin USP | 1.97 | 3.94 |
| Cherry Flavor FFS (223G12) | 0.20 | 0.40 |
| Menthol | 0.05 | 0.1 |
| Citric Acid (powder) | 0.75 | 1.5 |

A primary blend is prepared that contains sugar, corn syrup, sodium citrate, pectin and water. The primary blend is cooked to produce a residual moisture content of about 11% by weight. A secondary blend is prepared that contains dextromethorphan hydrobromide, phenylephrine hydrochloride, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 7

This example demonstrates a semi-solid chewable dosage form and its method of preparation in accordance with an embodiment of the invention. A 200 g batch is produced in this example. Each individual piece weighs 5 grams and contains 10 mg of dextromethorphan hydrobromide.

| | Formula | Batch |
|---|---|---|
| Ingredient | % by weight | 200 g |
| Sugar (powder) | 40.20 | 80.40 |
| Corn Syrup (dry) | 41.37 | 82.74 |
| Sodium Citrate (powder) | 0.39 | 0.78 |
| USP Citrus Pectin 200 (high methoxy) | 1.97 | 3.94 |
| Residual Water | 11.10 | 22.20 |
| Dextromethorphan Hydrobromide | 0.20 | 0.40 |
| Hydrolyzed Gelatin | 1.48 | 2.96 |
| Artificial sweetener (Sucralose) | 0.30 | 0.60 |
| Dodecalactone (1% in PG sol) | 0.01 | 0.02 |
| FD&C Red #40 | 0.01 | 0.02 |
| Glycerin USP | 1.97 | 3.94 |
| Cherry Flavor FFS (223G12) | 0.20 | 0.40 |
| Menthol | 0.05 | 0.10 |
| Citric Acid (powder) | 0.75 | 1.50 |

A primary blend is prepared that contains sugar, corn syrup, sodium citrate, pectin and water. The primary blend is cooked to produce a residual moisture content of about 11% by weight. A secondary blend is prepared that contains dextromethorphan hydrobromide, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

### Example 8

This example demonstrates a semi-solid chewable dosage form and its method of preparation in accordance with an embodiment of the invention. A 200 g batch is produced in this example. Each individual piece weighs 5 grams and contains 10 mg of loratadine.

| | Formula | Batch |
|---|---|---|
| Ingredient | % by weight | 200 g |
| Sugar (powder) | 40.20 | 80.40 |
| Corn Syrup (dry) | 41.37 | 82.74 |
| Sodium Citrate (powder) | 0.39 | 0.78 |
| USP Citrus Pectin 200 (high methoxy) | 1.97 | 3.94 |
| Residual Water | 11.10 | 22.20 |
| Loratadine | 0.20 | 0.40 |
| Hydrolyzed Gelatin | 1.48 | 2.96 |
| Artificial sweetener (Sucralose) | 0.30 | 0.60 |
| Dodecalactone (1% in PG sol) | 0.01 | 0.02 |
| FD&C Red #40 | 0.01 | 0.02 |
| Glycerin USP | 1.97 | 3.94 |
| Cherry Flavor FFS (223G12) | 0.20 | 0.40 |
| Menthol | 0.05 | 0.10 |
| Citric Acid (powder) | 0.75 | 1.5 |

A primary blend is prepared that contains sugar, corn syrup, sodium citrate, pectin and water. The primary blend is cooked to produce a residual moisture content of about 11% by weight. A secondary blend is prepared that contains loratadine, hydrolyzed gelatin, sucralose and dodecalactone (1% in propylene glycol solution). An additional blend is prepared that contains glycerin, colorants and flavorants. An acid solution is prepared that contains citric acid.

The secondary blend, additional blend and acid solution are combined with the primary blend to form the final blend. The final blend is mixed thoroughly. The final blend is transferred to a depositor hopper. From the depositor hopper, individual pieces are deposited into pre-formed plastic molds.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted y context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A semi-solid chewable dosage form comprising an active pharmaceutical ingredient selected from the group consisting of chlorpheniramine maleate, phenylephrine hydrochloride, and a combination thereof, a gelling agent, gelatin, sugar, a polyol, and a pH adjusting agent.

2. The semi-solid chewable dosage form of claim 1, wherein the active pharmaceutical ingredient is a combination of chlorpheniramine maleate and phenylephrine hydrochloride.

3. The semi-solid chewable dosage form of claim 2, wherein the gelling agent is pectin.

4. The semi-solid chewable dosage form of claim 2, wherein the gelatin is hydrolyzed gelatin.

5. The semi-solid chewable dosage form of claim 2, wherein the polyol is hydrolyzed starch hydrolysate.

6. The semi-solid chewable dosage form of claim 2, wherein the pH adjusting agent is sodium citrate and citric acid.

7. The semi-solid chewable dosage of claim 1, wherein the gelling agent is pectin in an amount from about 0.5% by weight to about 7% by weight, the gelatin is hydrolyzed gelatin in an amount from about 0.5% by weight to about 8% by weight, and the polyol is hydrolyzed starch hydrolysate in an amount from about 40% by weight to about 90% by weight.

8. A semi-solid chewable dosage form comprising an active pharmaceutical ingredient selected from the group consisting of chlorpheniramine maleate, phenylephrine hydrochloride, and a combination thereof, a gelling agent, gelatin, sugar, corn syrup, and a pH adjusting agent.

9. The semi-solid chewable dosage form of claim 8, wherein the active pharmaceutical ingredient is a combination of chlorpheniramine maleate and phenylephrine hydrochloride.

10. The semi-solid chewable dosage form of claim 9, wherein the gelling agent is pectin.

11. The semi-solid chewable dosage form of claim 9, wherein the gelatin is hydrolyzed gelatin.

12. The semi-solid chewable dosage form of claim 9, wherein the pH adjusting agent is sodium citrate and citric acid.

13. The semi-solid chewable dosage of claim 8, wherein the gelling agent is pectin in an amount from about 0.5% by weight to about 7% by weight, and the gelatin is hydrolyzed gelatin in an amount from about 0.5% by weight to about 8% by weight.

14. A semi-solid chewable dosage form comprising:
chlorpheniramine maleate;
phenylephrine hydrochloride;
pectin in an amount from about 0.5% by weight to about 7% by weight;
sugar in an amount from about 40% by weight to about 95% by weight;
hydrolyzed starch hydrolysate in an amount from about 40% by weight to about 90% by weight;
hydrolyzed gelatin in an amount from about 0.5% by weight to about 8% by weight;
sodium citrate in an amount from about 0.1% by weight to about 1% by weight; and
citric acid in an amount from about 0.5% by weight to about 3% by weight.

15. The semi-solid chewable dosage form of claim 14, further comprising glycerin in amount from about 0.5% by weight to about 5% by weight.

16. The semi-solid chewable dosage form of claim 15, further comprising dodecalactone.

17. The semi-solid chewable dosage form of claim 15, wherein the water content of the semi-solid dosage form is from about 8% by weight to about 15% by weight.

18. A semi-solid chewable dosage form comprising:
chlorpheniramine maleate;
phenylephrine hydrochloride;
pectin in an amount from about 0.5% by weight to about 7% by weight;
sugar in an amount from about 40% by weight to about 95% by weight;
corn syrup in an amount from about 40% by weight to about 90% by weight;
hydrolyzed gelatin in an amount from about 0.5% by weight to about 8% by weight;
sodium citrate in an amount from about 0.1% by weight to about 1% by weight; and
citric acid in an amount from about 0.5% by weight to about 3% by weight.

19. The semi-solid chewable dosage form of claim 18, further comprising glycerin in amount from about 0.5% by weight to about 5% by weight.

20. The semi-solid chewable dosage form of claim 19, wherein the water content of the semi-solid dosage form is from about 8% by weight to about 15% by weight.
